(19) United States
(12) Patent Application Publication
(10) Pub. No.: US 2019/0241927 A1
(43) Pub. Date: Aug. 8, 2019

(54) RAPID AND HIGHLY SENSITIVE BACTERIA CONTAMINATED WATER DETECTION AND PURIFICATION

(71) Applicants: Rachel Chang, Manhasset, NY (US); Ryan Thorpe, Manhasset, NY (US)

(72) Inventors: Rachel Chang, Manhasset, NY (US); Ryan Thorpe, Manhasset, NY (US)

(21) Appl. No.: 15/890,804

(22) Filed: Feb. 7, 2018

(51) Int. Cl. C12Q 1/04; C02F 1/72; C02F 1/00

(52) U.S. Cl. CPC C12Q 1/04; C02F 1/722; C02F 1/008; C02F 2305/023; C02F 2201/008; C02F 2303/04; C02F 2209/36

(57) ABSTRACT

Systems and methods for the detection and/or purification of bacteria contaminated water are disclosed. In an embodiment, a rapid, highly sensitive bacteria contaminated water detection and purification system is provided. A biosensor has a sensing region coupled between two terminals. The sensing region includes an oxidase enzyme immobilized on graphene. In the presence of bacteria respiration in contaminated water at the sensing region, a current is generated between the two terminals. A detector detects the generated current between the two terminals and generates a signal indicative of the presence of the bacteria in the contaminated water. A purification unit injects one or more fluids in the contaminated water to treat the contaminated water and obtain potable water. In one feature, bacteria associated with waterborne diseases can be detected rapidly with high sensitivity. In further embodiments, a handheld mobile system and array of biosensor devices are provided.

### BACKGROUND

### Technical Field

The field of the present invention relates to contaminated water detection and purification.

### Background

Waterborne diseases cause 3.4 million deaths annually. Worldwide, there exist 783 million individuals that do not have access to clean, potable water. The consumption and usage of contaminated water is the leading cause of the development of debilitating waterborne diseases such as Cholera, Shigellosis, E. coli, and Salmonella. See, “Facts and Statistics about Water and Its Effects.” The Water Project. N.p., 31 Aug. 2016. Web. 22 Jun. 2017. Yet, despite this severity, the traditional methods used to detect the presence of these bacteria can take 1-2 days, and have detection limits on the order of thousands of colony forming units (CFUs). See, Deshmukh, Rehan A., Kopal Joshi, Sunil Bhand, and Utpal Roy. “Recent Developments in Detection and Enumeration of Waterborne Bacteria: A Retrospective Minireview.” MicrobiologyOpen 5.6 (2016): 901-22. Web.

Biosensors have been used in a variety of biomedical applications to detect biological fluids, such as, blood glucose. Electrochemical biosensors can include amperometric biosensors, such as enzyme-based amperometric biosensors, that transduce a biochemical signal into an amperometric signal. See, G. Rocchitta et al., “Enyzme Biosensors for Biomedical Applications: Strategies for Safeguarding Analytical Performances in Biological Fluids,” Sensors 2016, 16, 780. Different types of biosensors have also been developed to detect toxic compounds like heavy metals, phenolic compounds, pesticides and herbicides in the environment. See, V. Nigam and P. Shukla, “Enzyme Based Biosensors for Detection of Environmental Pollutants—A Review,” J. Microbiol. Biotechno. (2015), 25 (11), 1173-1781.

Electrochemical biosensors detect materials through an electric signal that is created by an enzymatic reaction. Enzymes provide selectivity to the biosensor, which makes it a transducer for detection devices. Huang, Yinxi, et al. “Nanoelectronic Biosensors Based on CVD Grown Graphene.” Nanoscale, The Royal Society of Chemistry, 11 Jun. 2010. The enzyme layer is then able to catalyze the production of a current that generates a signal.

However, despite the widespread nature and severity of bacterial contamination in water, there is a lack of rapid and sensitive technology to detect these microorganisms much less purify contaminated water. As these pathogens have no known cure, a more sensitive, rapid detection device and purification method must be created in order to identify and eliminate microorganism presence in water sources to prevent contamination and the outbreak of pathogenic disease.

### BRIEF SUMMARY

Embodiments of the present invention overcome the above problems and deficiencies. The inventors disclose new and improved systems and methods for the rapid and sensitive detection and/or purification of bacteria contaminated water.

In an embodiment, a rapid, highly sensitive bacteria contaminated water detection and purification system is provided. The system includes a biosensor, a detector and a purification unit. The biosensor has a sensing region coupled between two terminals. The sensing region includes an oxidase enzyme immobilized on graphene. In the presence of bacteria respiration in contaminated water at the sensing region, a current is generated between the two terminals. The detector detects the generated current between the two terminals and generates a signal indicative of the presence of the bacteria in the contaminated water. The purification unit injects one or more fluids in the contaminated water to treat the contaminated water and obtain potable water.

In one feature, bacteria associated with waterborne diseases can be detected rapidly with high sensitivity.

In examples, the oxidase enzyme immobilized on graphene responds to analytes generated from respiration carried out by one of the following bacteria: S. typhi, V. cholerae, E. coli, or Shigella. In this way, bacteria associated with common waterborne diseases can be detected. In a further example the oxidase enzyme immobilized on graphene comprises at least one of the following oxidase enzymes: glucose oxidase, glycerol 3-phosphate oxidase, galactose oxidase, or lactate oxidase.

In one embodiment, an oxidase enzyme immobilized on graphene further includes pyrenebutryic acid (PBA) bonded onto the graphene.

In a further embodiment, a detector detects a generated current between two terminals and generates the signal indicative of the presence of the bacteria in the contaminated water in less than fifteen seconds. In a still further embodiment, the detector detects the generated current between the two terminals and generates the signal indicative of the presence of the bacteria in the contaminated water in about one second.

In a further embodiment, the detector detects the generated current between the two terminals and generates the signal indicative of the presence of the bacteria in the contaminated water at a detection limit of less than 100 CFUs per 100 ml. of water. In a still further embodiment, the detector detects the generated current between the two terminals and generates the signal indicative of the presence of the bacteria in the contaminated water at a detection limit of about one CFU per liter of water.

In one example, the detector is an ammeter that detects varying current indicative of the presence of bacteria respiration. In one feature, the ammeter detects varying current in an average range of about 0.001 to 0.012 nanoamperes (nA).

In one feature, the one or more fluids injected by the purification unit can be hydrogen peroxide and/or sodium hydroxide.

In another embodiment, a purification unit includes one or more microprocessor controlled ejection units to control the delivery of respective fluids into the contaminated water. In one example, each microprocessor controlled ejection unit has a microprocessor, a stepper motor coupled to an ejection unit that controls the amount of respective fluid injected into the contaminated water for treatment, and a servo-controller coupled between the microprocessor and the stepper motor, wherein the microprocessor generates signals to drive the servo-controller, and the servo-controller activates the stepper motor to control the ejection unit.

In a still further embodiment, a handheld mobile system is provided. A housing supports a battery, biosensor, detector and purification unit. The housing has a size suitable to be hand-held, whereby a user can hold the housing and insert the biosensor or the purification unit to contact the contaminated water.

In one embodiment, a biosensor device can detect bacteria contaminated water. The biosensor device has a graphene layer, an oxidase enzyme layer immobilized on the graphene layer, and a base that supports the graphene layer. A pair of conductive terminals are coupled to the oxidase enzyme layer at different locations. A current is generated between the terminals when bacteria respiration in the contaminated water produces analytes which contact enzymes in the oxidase enzyme layer.

In another embodiment, a system for detecting bacteria contaminated water includes an array of biosensor devices for detecting different respective bacteria in the contaminated water including the following bacteria: S. typhi, V. cholerae, E. coli, and Shigella. Each respective biosensor device in the array includes a respective graphene layer, a respective oxidase enzyme layer immobilized on the respective graphene layer, and a respective base that supports the respective graphene layer. A respective pair of conductive terminals are coupled to the respective oxidase enzyme layer at different locations. A current is generated between the pair of terminals when respiration by respective bacteria in the contaminated water produces analytes which contact enzymes in the respective oxidase enzyme layer.

Further embodiments, features, and advantages of the invention, as well as the structure and operation of the various embodiments of the invention are described in detail below with reference to accompanying drawings.

### BRIEF DESCRIPTION OF THE FIGURES

Embodiments are described with reference to the accompanying drawings. In the drawings, like reference numbers may indicate identical or functionally similar elements. The drawing in which an element first appears is generally indicated by the left-most digit in the corresponding reference number.

FIG. 1A is a diagram of a rapid, highly sensitive biosensor shown in a top view according to an embodiment of the present invention.

FIG. 1B is a diagram of the biosensor of FIG. 1A shown in a cross-sectional side view.

FIG. 2A is a diagram of a rapid, highly sensitive biosensor shown in a top view according to another embodiment of the present invention.

FIG. 2B is a diagram of the biosensor of FIG. 2A shown in a cross-sectional side view.

FIG. 3 is a diagram of a bacteria detection and water purification system according to an embodiment of the present invention.

FIG. 4 is a diagram of a handheld bacteria detection and water purification system according to another embodiment of the present invention.

FIG. 5 is a diagram of a rapid, multi-bacteria sensitive biosensor shown in a top view according to a further embodiment of the present invention.

FIGS. 6A, 6B, 7A-7D, 8A and 8B are graphs of results and statistical analysis obtained from operation of a rapid, highly sensitive biosensor and purification system in one example.

FIG. 6A shows amperage output of a control group of distilled water. FIG. 6B shows biosensor output in the presence of 1 CFU of S. aurantiaca in 100 ml. of water.

FIGS. 7A-7D display the average amperage in nanoamperes (nA) range output by different respective biosensors at different water volumes (100 ml. and 1000 ml.) and CFU levels (1, 5 and 10 CFUs). FIG. 7A shows biosensor output detecting the presence of Escherichia coli. FIG. 7B shows biosensor output detecting the presence of Vibrio fischeri. FIG. 7C shows biosensor output detecting the presence of Enterobacter aerogenes. FIG. 7D shows biosensor output detecting the presence of Sarcina aurantiaca.

FIG. 8A shows biosensor amperage output in the presence of 1 CFU of S. aurantiaca in 100 ml. of water. FIG. 8B shows the current output (amperage) following activation of a purification unit according to an embodiment.

### DETAILED DESCRIPTION OF EMBODIMENTS OF THE INVENTION

In embodiments, systems and methods are provided to rapidly and sensitively detect and/or purify water contaminated by bacteria. According to a feature in one embodiment, the inventors use enzymes to trigger an electrochemical response from a graphene biosensor to detect the presence of specific analytes released by bacteria during respiration in contaminated water. This can include rapid and highly sensitive detection of bacteria responsible for waterborne diseases including, but not limited to, one or more of the following bacteria: E. COLI, SHIGELLA, CHOLERA, AND SALMONELLA.

As used herein in embodiments with respect to contaminated water detection, the term “rapidly” means detecting bacteria presence in contaminated water in under about a minute. The term “highly sensitive” means detecting bacteria presence in contaminated water with a detection limit less than about 500 colony forming units (CFUs) per 100 milliliters (ml).

Embodiments refer to illustrations described herein with reference to particular applications. It should be understood that the invention is not limited to the embodiments. Those skilled in the art with access to the teachings provided herein will recognize additional modifications, applications, and embodiments within the scope thereof and additional fields in which the embodiments would be of significant utility.

In the detailed description of embodiments that follows, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

### Rapid, Highly Sensitive Biosensor Devices

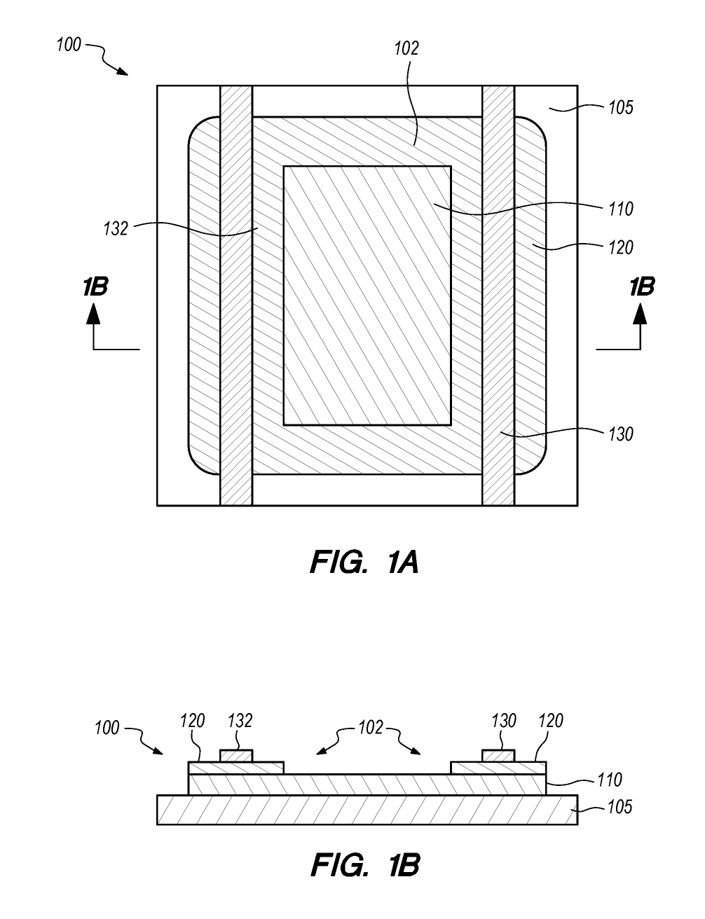

FIG. 1A is a diagram of a rapid, highly sensitive biosensor device 100 shown in a top view according to an embodiment of the present invention. FIG. 1B is a diagram of the biosensor 100 of FIG. 1A shown in a cross-sectional side view.

Biosensor device 100 can detect bacteria contaminated water rapidly with high sensitivity. Biosensor device 100 has a graphene layer 110 and an oxidase enzyme layer 120 immobilized on graphene layer 110. A base layer 105 supports graphene layer 110. Oxidase enzyme layer 110 can be immobilized in graphene layer 110 in a circle, square or other shape to form a sensing region 102. A pair of conductive terminals 130, 132 are coupled to oxidase enzyme layer 110 at different locations. As shown in FIGS. 1A and 1B, terminals 130 and 132 can be disposed on opposite sides of a sensing region 102. The sensing region 102 is where analytes can contact enzymes in oxidase enzyme layer 120.

In an example implementation, graphene layer 110 can be a thin layer of graphene that is highly conductive. For example, graphene layer 110 can be a two-dimensional allotrope of carbon that has a one atom thick hexagonal molecular structure to allow electrons to flow rapidly. This example is illustrative and a person skilled in the art given this description will recognize other types of graphene, graphite, or other highly conductive layers can be used.

In examples, oxidase enzyme layer 120 immobilized on graphene layer 110 responds to analytes generated from respiration carried out by one of the following bacteria: S. typhi, V. cholerae, E. coli, or Shigella. In this way, bacteria associated with common waterborne diseases can be detected.

In examples, oxidase enzyme layer 120 immobilized on graphene layer 110 can be one of the following oxidase enzymes: glucose oxidase, glycerol 3-phosphate oxidase, galactose oxidase, or lactate oxidase. These oxidase enzymes target analytes produced by respiration in each of the bacterium S. typhi, V. cholerae, E. coli, or Shigella. In one implementation, the enzyme immobilized onto layer 120 to detect S. typhi bacterium was a glucose oxidase (such as, glucose-6-P oxidase). In one implementation, the enzyme immobilized onto layer 120 to detect V. cholerae bacterium was a galactose oxidase (such as, α-D-glacsose-1,P). In one implementation, the enzyme immobilized onto layer 120 to detect E. coli bacterium was a lactate oxidase (such as, d-lacate oxidase). In one implementation, the enzyme immobilized onto layer 120 to detect Shigella bacterium was a glycerol phosphate oxidase (such as, glycerol 3-phosphate oxidase).

In one embodiment, any of these oxidase enzymes can be immobilized on graphene layer 110 by utilizing 1-pyrenebutryic acid (PBA) as a linker molecule. These oxidase enzymes and immobilization examples are illustrative and a person skilled in the art given this description will recognize other types of oxidase enzymes and immobilization can be used depending on a particular analyte being detected from bacteria respiration.

Base 105 can be a polyethylene terephthalate (PET) film, plastic, or other material that can support graphene layer 110 and provide structural stability for biosensor 100. Base 105 can be flexible or rigid depending upon a particular application. Base 105 can also be mounted on a further support element or surface as desired. These examples are illustrative and a person skilled in the art given this description will recognize other types of material and arrangements can be used to provide a support base in biosensor 100.

In one feature, bacteria associated with waterborne diseases can be detected rapidly with high sensitivity by biosensor device 100. This rapid and highly sensitive detection is described further with respect to the operation of biosensor device 100.

### Biosensor Device Operation

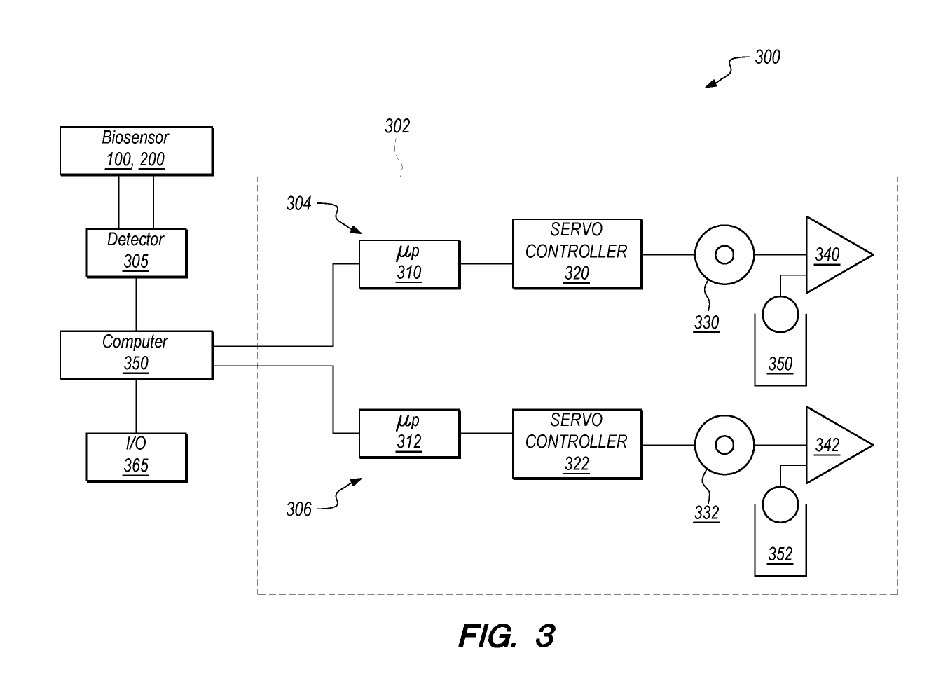

In operation, biosensor device 100 is placed in contact with water to be evaluated for bacterial contamination. An electrical current is generated between terminals 130, 132 when bacteria respiration in the contaminated water produces analytes which contact enzymes in oxidase enzyme layer 120. A detector, such as an ammeter, can be coupled to the terminals 130, 132 to detect the current for display or storage, or for output to a computer for further processing and display (as described in further detail below with respect to detector 305 and computer 360 in FIG. 3). For example, the ammeter can apply a potential across terminals 130, 132 and detect an electrical current generated when analytes contact enzymes in oxidase enzyme layer 120.

According to further features, biosensor device 100 coupled to a detector can detect bacteria in contaminated water rapidly and with high sensitivity. For example, the sensitivity of the oxidase enzyme layer 120 and high conductivity and electron mobility of graphene layer 110 allows an output to be provided from biosensor device 100 to a detector rapidly with high sensitivity. In one example, the detector is an ammeter that detects varying current indicative of the presence of bacteria respiration. In one feature, an ammeter detects varying current in an average range of about 0.001 to 0.012 nanoamperes (nA).

With respect to rapid detection, the ammeter can receive output from biosensor device 100, detect a varying current between the two terminals 130, 132, and generate a signal indicative of the presence of the bacteria in the contaminated water in less than fifteen seconds. In a still further embodiment, the ammeter detects the varying current between the two terminals 130, 132 and generates a signal indicative of the presence of the bacteria in the contaminated water in about one second.

With respect to high sensitivity, the ammeter detects the varying current between the two terminals 130, 132 and generates a signal indicative of the presence of the bacteria in the contaminated water at a detection limit of less than 100 CFUs per 100 ml. of water. In a still further embodiment, the ammeter detects the generated current between the two terminals 130, 132 and generates the signal indicative of the presence of the bacteria in the contaminated water at a detection limit of about one CFU per liter of water.

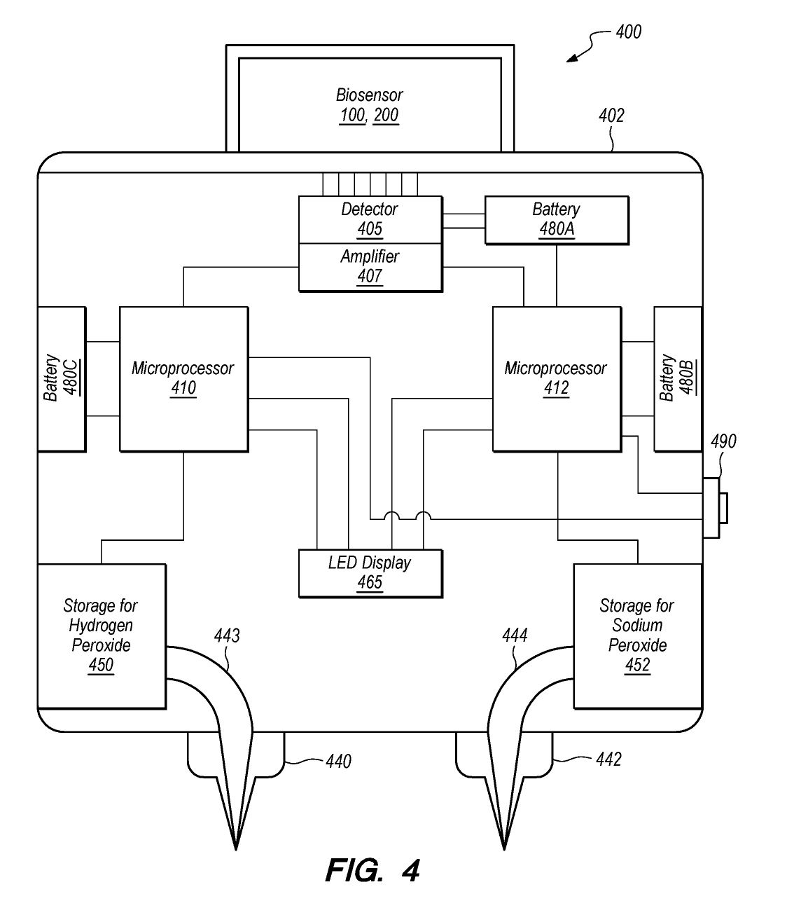

Rapid and highly sensitive detection is described in further detail below with respect to FIGS. 3-4 and an example study and results.

### Further Biosensor Device Embodiments

The above description of biosensor device 100 as a single sensor is illustrative for clarity and not intended to be limiting. In other implementations, biosensor device 100 can include additional layers for protection or support depending upon a particular application. Different arrangements and packaging can be used as well as would be apparent to a person skilled in the art given this description.

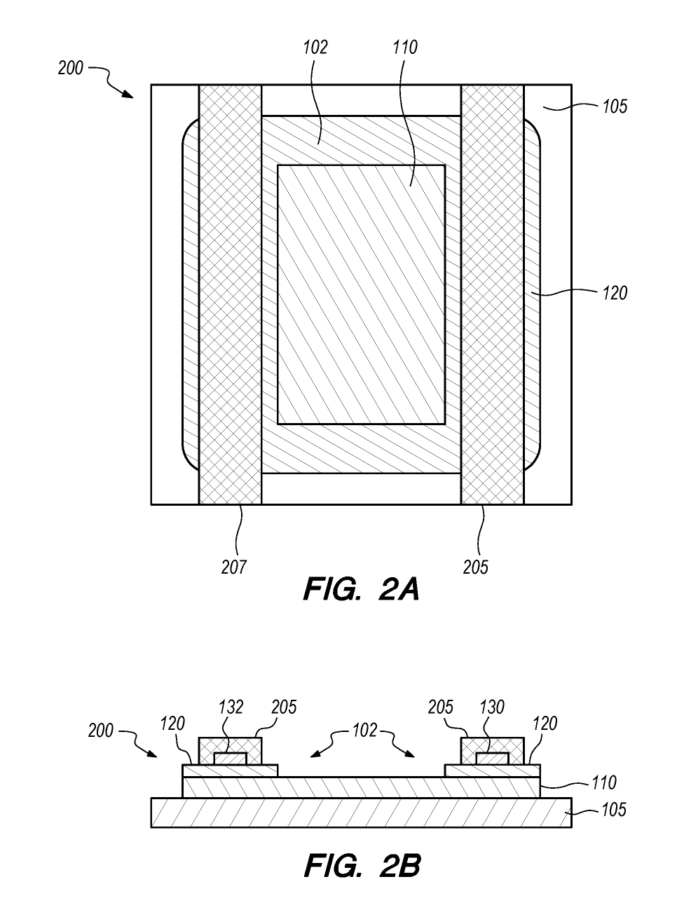

In a further feature a carbon epoxy can be provided on terminals of biosensor 100 to further protect regions of the sensor device from exposure to water, air or other environment conditions. FIG. 2A is a diagram of a rapid, bacteria sensitive biosensor 200 shown in a top view according to another embodiment of the present invention. FIG. 2B is a diagram of the biosensor 200 of FIG. 2A shown in a cross-sectional side view. Biosensor 200 has carbon black epoxy layers 205 and 207 provided on respective terminals 130 and 132 of biosensor 100. These carbon black epoxy layers 205 and 207 can help protect biosensor 200 when placed in rugged environments such as a water pipe, stream, or other area with a strong water current or varying range of temperature or pressure.

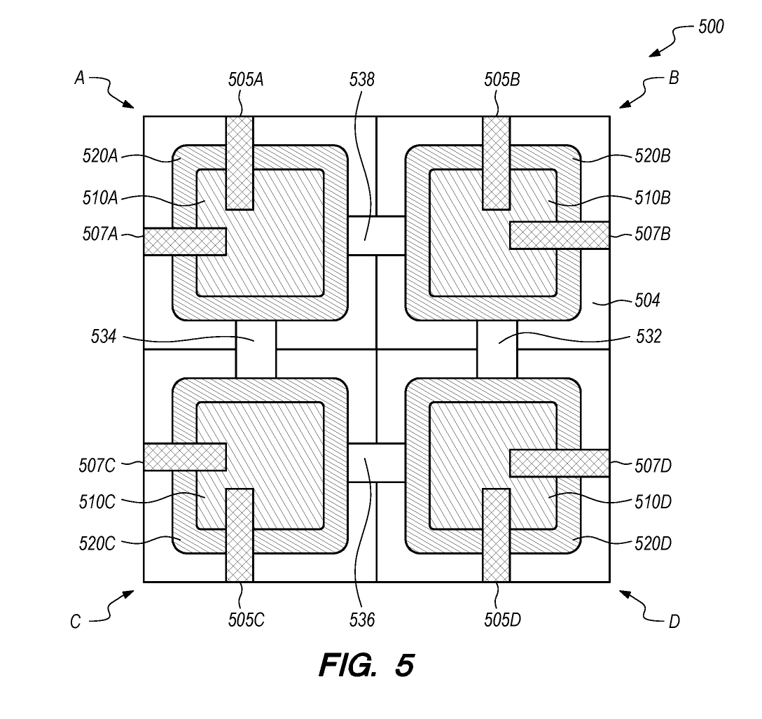

In further embodiments, biosensor devices 100, 200 can be arranged as an array of biosensor devices to allow detection of multiple types of bacteria. Examples are described further below with respect to FIG. 5.

### Rapid and Sensitive Bacteria Detection and Water Purification System

According to a feature, embodiments are provided for the rapid and sensitive detection and purification of bacteria contaminated water.

FIG. 3 shows a diagram of a rapid and sensitive bacteria detection and water purification system 300 according to an embodiment of the present invention. System 300 includes one or more biosensors 100, 200 and a purification unit 302. A detector 305, such as an ammeter, is coupled to receive an input from terminals at biosensor 100 or 200. A computer 360 having an input/output interface 365 is coupled to receive an output signal from detector 305. Computer 360 can also be coupled to provide control signals to microprocessor controlled ejection units 304, 306.

In the embodiment shown in FIG. 3, purification unit 302 includes two microprocessor controlled ejection units 304, 306 to control the delivery of respective fluids into the contaminated water. In one feature, the fluids injected by purification unit 302 can be hydrogen peroxide and/or sodium hydroxide. A storage reservoir 350 contains hydrogen peroxide. A storage reservoir 352 contains sodium hydroxide.

As shown in FIG. 3, microprocessor controlled ejection assembly 304 has a microprocessor 310, a servo-controller 320, stepper motor 330 and ejection unit 340. Microprocessor 310 is coupled to servo-controller 320. Servo-controller 320 is coupled to stepper motor 330. Stepper motor 330 is coupled to ejection unit 340.

Ejection unit 340 controls the amount of respective fluid injected into the contaminated water for treatment. For example, ejection unit 340 may have a valve that opens or closes in response to movement of stepper motor 330 to regulate an amount of hydrogen peroxide that passes from storage reservoir 350 through a pipe or tube into the contaminate water for treatment. In operation, microprocessor 310 generates control signals to drive servo-controller 320. Servo-controller 320 activates stepper motor 330 to control ejection unit 340 according to the servo signals from servo-controller 320.

Microprocessor controlled ejection assembly 306 is similar to microprocessor controlled ejection assembly 304 except it controls the delivery of sodium hydroxide instead of hydrogen peroxide. Microprocessor controlled ejection assembly 306 has a microprocessor 312, a servo-controller 322, stepper motor 332 and ejection unit 342. Microprocessor 312 is coupled to servo-controller 322. Servo-controller 322 is coupled to stepper motor 332. Stepper motor 332 is coupled to ejection unit 342.

Ejection unit 342 controls the amount of respective fluid injected into the contaminated water for treatment. For example, ejection unit 342 may have a valve that opens or closes in response to movement of stepper motor 332 to regulate an amount of hydrogen peroxide that passes from storage reservoir 352 through a pipe or tube into the contaminate water for treatment. In operation, microprocessor 312 generates control signals to drive servo-controller 322. Servo-controller 322 activates stepper motor 332 to control ejection unit 342 according to the servo signals from servo-controller 322.

Microprocessors 312, 314 can be programmed, set up, or controlled in realtime to provide control signals. These control signals can include control signals, such as, start, stop, run or calibrate. Control buttons (not shown) can also be added to initiate operations in each of the microprocessors 312, 314. In a further example, computer 360 can be coupled to each of microprocessors 312, 314 to program the microprocessors 312, 314 with control instructions. Computer 360 can be coupled to each of microprocessors 312, 314 to control the microprocessors 312, 314 in realtime with control instructions.

Computer 360 can be any type of computing device including, but not limited to, a desktop computer, laptop computer, tablet device, mobile device, controller or smartphone. Input/output interface 365 can be a display, keyboard, touchscreen, and/or other peripherals to provide control inputs and/or outputs.

### Handheld Bacteria Detection and Water Purification Device

In a still further embodiment, a handheld rapid and sensitive bacteria detection and water purification device is provided. FIG. 4 shows a handheld mobile rapid and sensitive bacteria detection and water purification device 400 according an embodiment. Device 400 includes a housing 402 suitable to be held by user. For example, housing 402 can be shaped to fit a human hand for carrying or for holding during use in contaminated water detection and treatment. Housing 402 can also be fitted with a handle to ease carrying and holding provided with for a handheld device. One or more batteries 480A-480C are also provided to provide power during remote use.

Housing 402 supports one or more batteries 480A-C, a biosensor 100 or 200, detector 405, amplifier 407, and a purification unit made up of two microprocessors 410, 412, piping 443, 444, ejection units 440, 442 and storage reservoirs 450, 452. Housing 402 can also include a display 465. The housing has a size suitable to be hand-held, whereby a user can hold the housing and insert a biosensor 100 or 200 to contact the contaminated water or allow ejection units 440, 442 to insert fluids to treat the contaminated water.

As shown in FIG. 4, biosensor 100, 200 is coupled through its terminals to detector 405, such as an ammeter. Detector 405 outputs a signal to amplifier 407 which sends an amplified output signal to microprocessors 410, 412. One or both of microprocessors 410, 412 can then output data signals to display information on display 465. The displayed information can include for example, an indication of the detection of the presence or absence of bacteria in contaminated water by biosensor 100, 200. Other information can also be displayed such as the detected current values or the average detected current value over a period of time such as one to 15 seconds. Batteries 480A-C can be a single battery or multiple batteries in housing 402. In one example, batteries 480A-C are rechargeable. Batteries 480A-C may be electrically coupled to detector 405, amplifier 407, microprocessors 410, 412, and display 465 to provide power.

When microprocessors 410, 412 receive an amplified output signal from amplifier 407 indicative of the presence of bacteria respiration in contaminated water, one or both of microprocessors 410, 412 can initiate treatment of the water. Microprocessors 410, 412 can initiate treatment of the water automatically when bacteria is detected or in response to a user pressing a button 490 coupled to microprocessors 410, 412 to manually start water treatment. To treat (also called purify) the contaminated water, microprocessor 410 sends a control signal to open or close a valve (not shown) coupled to a storage reservoir 450 so that a fluid therein (such as hydrogen peroxide) can flow through piping 443 to a nozzle or port at an ejection unit 440 into the contaminated water. Similarly, to treat the contaminated water, microprocessor 412 sends a control signal to open or close a valve (not shown) coupled to a storage reservoir 452 so that a fluid therein (such as sodium hydroxide) can flow through piping 444 to a nozzle or port at an ejection unit 442 into the contaminated water.

In a further embodiment, a communication port or interface (not shown) can be coupled to one or more microprocessors 410, 412 to allow wired or wireless data communication with a remote control device such as an application on mobile phone or smart watch.

### Array of Biosensor Devices

While biosensors 100, 200 were each described with respect to a single sensor arrangement for clarity, the present invention is not so limited. Biosensors 100, 200 can also be used in an array of biosensors made up of multiple biosensors 100, 200 arranged in a line, circle, rectangle, or other pattern or arrangement depending upon a particular application or design need.

FIG. 5 shows a diagram of a rapid, multi-bacteria sensitive biosensor array 500 shown in a top view according to a further embodiment of the present invention. In this example, a system for detecting bacteria contaminated water 500 includes an array of four biosensor devices 505A-505D in a quadrant configuration for detecting different respective bacteria in the contaminated water including the following bacteria: S. typhi, V. cholerae, E. coli, and Shigella. Each respective biosensor device 505A-505D in the array includes a respective graphene layer 510A-D, a respective oxidase enzyme layer 520A-D immobilized on the respective graphene layer 510A-D, and a respective base such as a PET film that supports the respective graphene layer 510A-D. In this example, four PET film channels 532, 534, 536, 538 are in between respective pairs of the four biosensor devices 505A-505D. A hydrophobic substance layer 504 is provided on regions outside sensing regions of the biosensor devices 505A-505D.

Each biosensor device 505A-505D has a respective pair of conductive terminals 505A-D, 507A-D coupled to the respective oxidase enzyme layer 520A-D at different locations. In each biosensor device 505A-505D, a current is generated between the pairs of terminals 505A-D, 507A-D when respiration by respective bacteria in the contaminated water produces analytes which contact enzymes in the respective oxidase enzyme layer. In this way, by providing four different oxidase enzyme layers 520A-D sensitive to different bacteria, S. typhi, V. cholerae, E. coli, and Shigella, the four biosensor devices 505A-505D can detect different respective bacteria in the contaminated water.

In examples, biosensor 500 can also be used in place of biosensor 100 in any of the systems 300, 400 described above.

### Additional Features and Advantages

Overall, embodiments herein have important implications for the developing world and other areas in need in terms of water contamination monitoring and purification. The rapid and sensitive bacteria detection and water purification systems 300, 400 described herein can be implemented into a drinking source in order to consistently monitor the presence of E. coli, Shigella, Salmonella, and Cholera in the water. If the biosensor device(s) 100, 200, 500 identify that there exists bacteria in the water, purification unit 302 can be implemented in order to add the chemical agents necessary to generate hydroxyl radicals in order to purify water contaminated with the bacteria. Handheld rapid and sensitive bacteria detection and water purification system 400 allows even more remote deployment and ease of use to access drinking sources of water for testing and treatment.

These results are fundamental to the advancement of water sanitation throughout the world, in the developing and developed world alike. In the developing world, many women and children spend upwards of 6 hours each day collecting water. UN Water. (2013). UN-Water factsheet on water and gender, World Water Day 2013. Embodiments herein including systems 300, 400 can alleviate this issue, allowing women and children to receive an education, by providing a user-friendly, labor-saving sanitation system for drinking water. Similarly, access to safe water in the developing world has the potential to provide almost $32 billion dollars in economic benefits. Deshmukh, Rehan A., Kopal Joshi, Sunil Bhand, and Utpal Roy. “Recent Developments in Detection and Enumeration of Waterborne Bacteria: A Retrospective Minireview.” MicrobiologyOpen 5.6 (2016): 901-22. Web.

Rapid and sensitive bacteria detection and water purification systems 300, 400 can be applied to industrial settings as well. Biosensors 100, 200, 500 can be formatted into a cylindrical structure in order to be placed in a pipe of flowing water. A biosensor can also be covered in electrically conductive carbon black epoxy, which will prevent high volumes of water at high speeds to reduce stress or degradation of material at the terminals. This device implementation would allow for countries and regions to constantly monitor bacterial presence and reduce the threat that waterborne pathogens have on the world.

### Further Discussion, Example Study and Results

The following discussion provides further explanation and is not intended to limit the present invention.

The inventors carried out a study of several implementations of a rapid, highly sensitive water detection and purification system 300 with biosensor device(s) 100. The results of this study and results described here are illustrative and not intend to be limit the present invention. The study aimed to create various graphene-based biosensors as described herein in order to recognize minute levels of specific bacteria in a rapid time frame and fabricate a mechanized purification system to eliminate this bacterial presence.

In a study carried out by the inventors, the inventors constructed biosensors using graphene and enzymes in order to detect analytes produced by waterborne pathogens. Graphene is a two-dimensional allotrope of carbon that has a one atom thick hexagonal molecular structure with benzene rings, which allows electrons to flow rapidly through the thin material. See, Fuente, Jesus De La. “Graphene—What Is It?” Graphenea. N.p., n.d. Web, 25 Mar. 2017. With this high conductivity and electron mobility, it serves as an ideal electrode for the creation of biosensors. Putzbach's (2013) study determined that enzymatic biosensors can be created through the incorporation of nanomaterials. Putzbach, William, and Niina Ronkainen (2013) “Immobilization Techniques in the Fabrication of Nanomaterial-Based Electrochemical Biosensors: A Review.” Sensors 13.4: 4811-840. Web.

The inventors for the first time used biosensors for bacterial analyte detection in contaminated water This study included utilizing oxidase enzymes and graphene electrodes in converting bacterial respiratory products into a detectable current. In one test, the enzymes Lactate Oxidase, Glucose Oxidase, Glycerol Oxidase, and Galactose Oxidase were immobilized by utilizing a linker molecule, 1-pyrenebutyric acid (PBA). This acid is a linker molecule that acts as a bridge between the enzyme and the graphene through π-stacking. PBA has an aromatic molecular structure, containing benzene rings that non-covalently stabilize atop the graphene hexagonal rings. These enzymes are immobilized onto the graphene through π-π interactions. π-interactions are a type of stable, non-covalent bond that can exist between π-systems, allowing for interactions between aromatic molecules and protein structures to exist. See, Huang, Yinxi, et al. “Nanoelectronic Biosensors Based on CVD Grown Graphene.” Nanoscale, The Royal Society of Chemistry, 11 Jun. 2010. π-interactions were analyzed in their applications in enzyme immobilization. π-π interactions occur between 2 aromatic molecules, or 2 molecules that contain hexagonal benzene rings in their molecular structure. The two benzene rings, or the π rings, are able to stack up one another and align themselves to stably bind the molecules together. The linker molecule then bonds with the enzyme through an amide bond that occurs between the amine group of the enzyme and the pyrene group of the PBA, a process known as amidation. See, Kathyavini, Nagaraju (2015) A Review on Protein Functionalized Carbon Nanotubes: it. pag. Journal of Applied Biomaterials & Functional Materials. Web. Labroo, Pratima, and Yue Cui (2013) “Flexible Graphene Bio-nanosensor for Lactate.” Biosensors and Bioelectronics 41: 852-56. Web. This safely and securely immobilizes the enzymes onto the graphene without damaging either structure, allowing the enzymes to serve as a permanent transducer on the biosensors.

Equally as important as the detection of bacteria is the elimination of bacteria from water sources. Hydrogen peroxide and sodium hydroxide can be utilized in order to eliminate organic material in water. When hydrogen peroxide and sodium hydroxide are added to water, the water will experience a raise in pH that contributes to a process known as hydrogen peroxide bleaching, a process by which hydroxyl radicals are formed. These radicals have the potential to degrade lipids and proteins. As bacterial pathogens are made of lipids and proteins, these radicals can eliminate bacterial presence. See, Kommineni, Suril (2016) “Advanced Oxidation Processes.” SpringerBriefs in Molecular Science Novel Catalysts in Advanced Oxidation of Organic Pollutants: 23-34. Web. This would effectively purify water and make it safe for consumption, as the only byproducts of this reaction are carbon dioxide and water.

Waterborne diseases cause 3.4 million deaths annually, concentrated in countries lacking sanitary water. Conventional methods, such as PCR, can be costly, take several days, and have detection limits up to 1,000 CFUs. Thus, the purpose of this study was to engineer a system that could efficiently detect and purify bacterial presence in a more rapid time frame and a lower detection threshold. Graphene was utilized to create biosensors through the immobilization of enzymes that target analytes released during respiration of Salmonella, Shigella, Cholera, and E. coli. When these bacteria respire, they produce analytes that, when in contact with the enzymes, produce a varying electric current that can be read by an ammeter to determine the presence of bacteria. The sensors were able to detect the presence of at least 1 CFU of bacteria in 1 L of water instantaneously. A mechanized approach was then taken to purify contaminated water samples through the use of Arduino microcontrollers. This system was successful in detecting minute levels of bacteria in a rapid time frame and purifying the water of pathogens. These detection and purification systems can be used in conjunction to decrease the threat of waterborne disease that exists in the world today.

In this study, all chemicals were purchased from Sigma Aldrich, CVD graphene and transfer tape were purchased from the Graphene Supermarket, ammeter and counter electrodes were purchased from eDAQ, bacteria and culturing supplies were purchased from Carolina Biological and purification unit components were purchased via Amazon and Robot Geek.

Phase 1:

A graphene biosensor (such as an example biosensor device 100) was created in order to rapidly and sensitively detect bacterial contamination in water by S. typhi, V. cholerae, E. coli, and Shigella through the isolation of graphene and immobilization of specific enzymes that target the analytes produced by each bacterium.

In this study, E. coli K12 was used as a model organism for E. coli O157:H7, Enterobacter aerogenes modelled Shigella dysenteriae, Sarcina aurantiaca was used as a model for Salmonella typhi, and Vibrio fischeri modelled Vibrio cholerae, because of the metabolic and respiratory parallels between the organisms. Firstly, both E. coli K12 and E. coli O157:H7 are gram-negative, rod-shaped, and both perform mixed acid anaerobic and aerobic respiration. It is through respiration that both strains of E. coli can generate D-lactate. See, Förster, Andreas H., and Johannes Gescher. “Metabolic Engineering of Escherichia Coli for Production of Mixed-Acid Fermentation End Products.” Frontiers in Bioengineering and Biotechnology, Frontiers Media S.A., 2014. Second, Sarcina aurantiaca was used as a model organism for S. typhimurium as both organisms have CrsA-monitored respiration pathways by which glucose-6-P is produced. See, Ali, Mohamed M (2014) “Fructose-Asparagine Is a Primary Nutrient during Growth of Salmonella in the Inflamed Intestine.” PLOS. PLOS Pathogens 25 Mar. 2017 Third, Vibrio fischeri was used as a model organism for Vibrio cholerae because both organisms belong to the Vibrio genus, and both organisms produce α-D-Galactose-1,P as a byproduct of glycolysis. Finally, Enterobacter aerogenes was used as a model organism for Shigella dysenteriae because both organisms produce glycerol during respiration. See, Müller, Ulrike Maria, Liang Wu, Lourina Madeleine Raamsdonk, Aaron Adriaan Winkler, and Dsm Ip Assets B.V (2008) “Acetyl-coa Producing Enzymes in Yeast.” Google Books. BibTex, Web. 21 Nov. 2016.

Graphene Isolation

The inventors isolated graphene in two manners in order to analyze the efficiency of more cost effective methods.

First, CVD Graphene was purchased on a Ni/SiO2/Si wafer from Graphene Supermarket. In order to isolate the graphene layers, the nickel and silicon/silicon dioxide layers were removed. To accomplish this, thermal release tape was adhered onto the graphene with applied pressure in order to remove air bubbles and increase the continuity of the graphene layer. It was then exposed to 30 mL of deionized water and ultrasonicated for 180 seconds. The ultrasonicator produces 20 kHz of sound waves that intervene between the layers of the wafer, breaking the intermolecular interactions and removing the silicon/silicon dioxide layers from the graphene wafer. Then, in order to remove the nickel layer on the wafer, nickel etching was performed using a 0.1 M solution of ferric chloride. The ferric chloride was warmed to 55° C. on a hot plate underneath a fume hood. The graphene with tape was then placed in the solution and stirred for approximately 10 minutes, removing the nickel layer. The graphene on the tape was then removed from the solution and rinsed with deionized water (The Graphene Supermarket). These procedures were repeated for the isolation of graphene for the 3 remaining graphene wafers. At this point, only graphene remained on the thermal release tape. In order to transfer it onto a flexible substrate, the thermal release tape was adhered onto a PET film and placed on a hot plate set to 130° C., the release point of the tape, for 10 seconds (The Graphene Supermarket). When at 130° C., the tape released the graphene molecules onto the plastic substrates.

Graphite-Flake Dispersion

Despite CVD graphene's ability to form a highly functioning electrode, the inventors recognized a graphite-flake dispersion between oil and water can also be utilized in order to reduce overall costs of the biosensor. Graphite from a standard graphite pencil was rubbed against a square of thermal release tape, which was then adhered to another piece of thermal release tape. 15 mL of deionized water was added to a beaker, and 15 mL of oil was pipetted on top of the water. The two substances remained separated due to the differences of polarity of water and oil. These two substances formed an interface caused by the surface tension that arose from the hydrogen bonding of water. The graphite thermal release tape was then placed in the oil and water interface and ultrasonicated for 3 minutes, which would create an emulsification. The thermal release tape was then removed and a single-layer of graphite, or graphene, existed on the plastic tape films. See, Tang, Zhihong (2010) “Exfoliation of Graphene from Graphite and Their Self-Assembly at the Oil-Water Interface.” Researchgate. Tsinghua University, Web 12 Apr. 2017.

Graphene Functionalization

The graphene on the plastic films, both from CVD and graphite origin, were submerged by the inventors into a 5 mM solution of 1-pyrenebutyric acid dissolved in DMSO and left at room temperature. After 2 hours, the substrates were removed and rinsed with distilled water. This bonded the PBA onto the graphene through π-π interactions. See, Kathyayini, Nagaraju (2015) A Review on Protein Functionalized Carbon Nanotubes: n. pag. Journal of Applied Biomaterials & Functional Materials. Web. Glucose Oxidase, Glycerol 3-Phosphate Oxidase, Galactose Oxidase, and Lactate Oxidase, purchased from Sigma-Aldrich in anhydrous form, were immobilized onto the graphene. In order to immobilize the enzymes onto the graphene, the inventors added 24 μL of distilled water to the anhydrous molecules of the enzyme and the solution was ultrasonicated for 180 seconds. This was repeated for the remaining enzymes. One aliquot of each enzyme was then micro-pipetted onto one of the graphene substrates and left at 7° C. overnight to immobilize the enzymes onto the graphene. Conductive paint terminals were then applied onto opposite, parallel sides of the graphene biosensor.

Testing

In order to test the functionality of these biosensors, the bacteria Enterobacter aerogenes, Vibrio fischeri, E. coli K12, and Sarcina aurantiaca were cultured. All bacteria were purchased from Carolina Biological and cultured onto LB agar plates using sterile techniques. An isolation streaking technique was performed in order to make colonies visible on the agar plates for testing (Carolina Biological).

In order to test the sensitivity of the biosensor, differing colony counts were inoculated into various water Photo by Competition Entrant volumes. 0, 1, 5 and 10 colonies of each bacteria were incorporated into 100 mL and 1 L of distilled water. The control group contained solely distilled water, while the variable groups contained a specific number of bacterial CFUs in one of the water volumes. A 5 μL sample was then micro-pipetted into the center of the designated biosensor for testing bacterial presence with an eDAQ ammeter (EPU 357 Conductivity isoPod) through platinum/titanium electrode wires. This ammeter measures the current in nanoamps. If bacteria are present in the sample, they are respiring and releasing analytes onto the biosensor that would free electrons and generate a changing electric signal. Based on the values outputted by the ammeter, the presence of bacteria will be determined. See, Labroo, Pratima, and Yue Cui (2013) “Flexible Graphene Bio-nanosensor for Lactate.” Biosensors and Bioelectronics 41: 852-56. Web.

Phase 2: A mechanized purification unit was created by inventors utilizing an advanced hydrogen peroxide reaction. Sodium hydroxide and hydrogen peroxide can be ejected into a water source in order to generate hydroxyl radicals that can eliminate organic material in water. The OH— groups of sodium hydroxide cause hydrogen peroxide to denature into 2OH. (Hydroxyl Radicals), due to the raise in pH, at faster rates. When hydroxyl radicals come into contact with organic material, it creates safe byproducts of carbon dioxide and water, forming after the degradation of organic material and the neutralization of the reaction with sodium bicarbonate. See, Swaminathan, Meenakshiundaram, Manickavachagam Muruganandham, and Mika Sillanpaa. “Advanced Oxidation Processes for Wastewater Treatment.” International Journal of Photoenergy (2013): 1-216. Web.

A mechanized unit was created using an Arduino microprocessor to regulate the implementation of chemicals into water sources. The Arduino Zero was then connected to a Darrington Board using jumper cables. The pins of the Arduino (D8-D11) were connected to the corresponding number of the Darrington Board (INT1-INT4). This allowed for the Arduino to power the board as well as upload the rotation code onto it. The Arduino and the Darrington Board were then mounted onto a RobotGeek workbench using metal wiring. A stepper motor was attached to the Darrington Board using 5 lead adjoined wires.

A stepper motor code provided with the Arduino module was manipulated to fit the parameters of the purification unit. Once this code was uploaded, a spring was attached to a micropipette via adhesive glue. The micropipette was then elevated off the RobotGeek Workbench using a stack of 4, 3.5×2.7×0.3 cm plastic prisms and adhered using glue. This system would allow for the safe uptake and ejection of purification chemicals into water.

This process was repeated in order to mount two micropipettes onto the workbench. The micropipettes were filled with sodium hydroxide (0.5M, 10 μL) and hydrogen peroxide (3% 10 μL). The activation of the Arduino units led to the ejection of these chemicals into a water source. After ejection, sodium bicarbonate was added to water in order to disrupt the functioning of the hydroxyl radicals and was filtered out of water with a coffee filter. See, Kommineni, Suril (2016) “Advanced Oxidation Processes.” SpringerBriefs in Molecular Science Novel Catalysts in Advanced Oxidation of Organic Pollutants: 23-34. Web. In order to test the functionality of the device, bacteria were inoculated into a water source, and a sample of the water was placed on the biosensors in order to confirm the presence of bacteria in water. Next, the purification unit was activated, leading to the ejection of chemicals loaded in the micropipettes. Samples of water were then removed from the decontaminated water source in order to confirm that the purification unit had success in eliminating bacterial presence in water. This was accomplished by placing volumes of water onto the biosensor and monitoring the current with the EDAQ ammeter. A flat, unchanging current graph designated that the system had success in eliminating bacterial presence in water as shown in the results below.

Results

Phase 1:

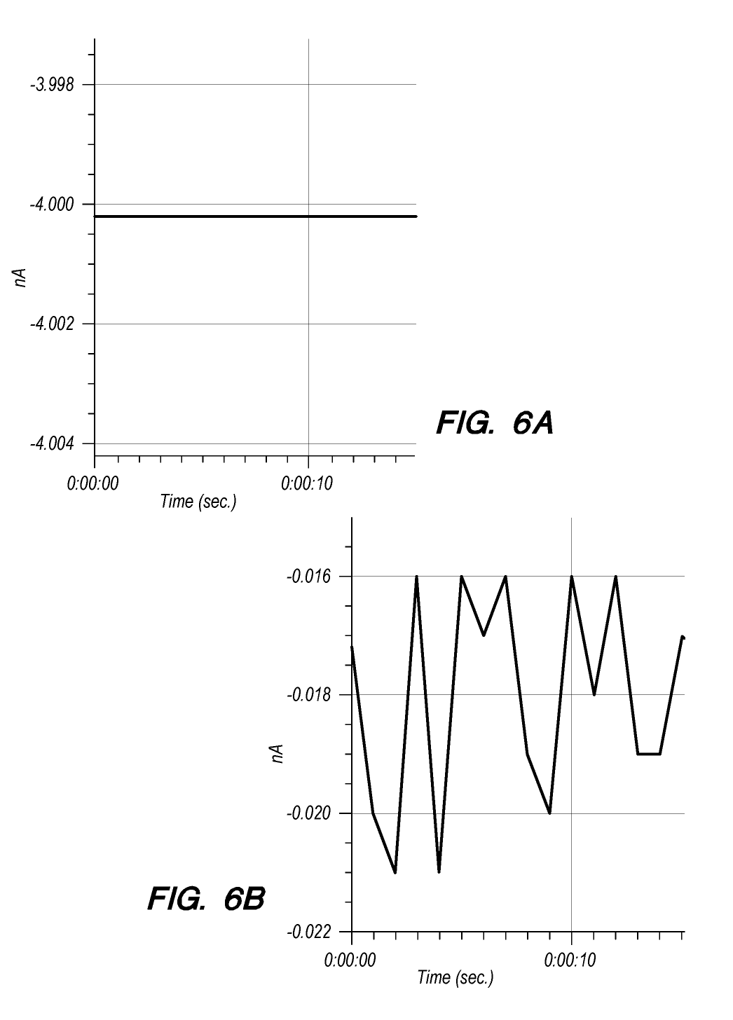

During testing, each of the biosensors were exposed to a sample of contaminated water for 15 seconds. Samples contained varying volumes of water (100 mL and 1000 mL) and varying CFU counts (0, 1, 5, and 10 CFU). The graph in FIG. 6A shows the output of a sample of solely distilled water. As there are no bacteria present in a sample, no analytes are freeing electrons, which creates this unchanging current output. However, when bacteria are present as seen in the graph of FIG. 6B, the bacteria releases analytes that undergoes a series of chemical reactions to generate a changing current. A changing current output represents that bacteria are present in the sample. The range of the current values that were outputted by the eDAQ ammeter was calculated for each of these trials.

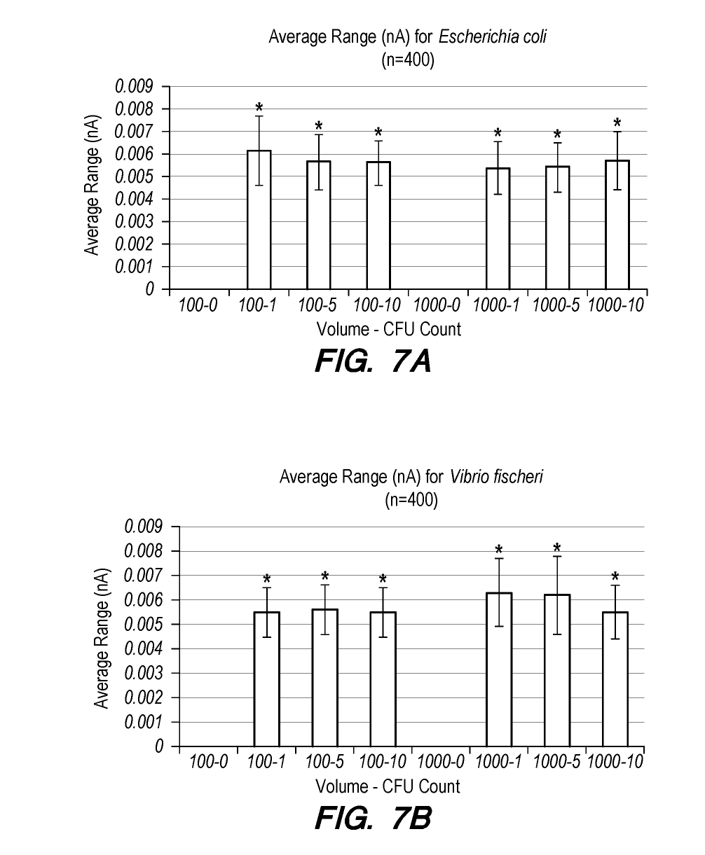

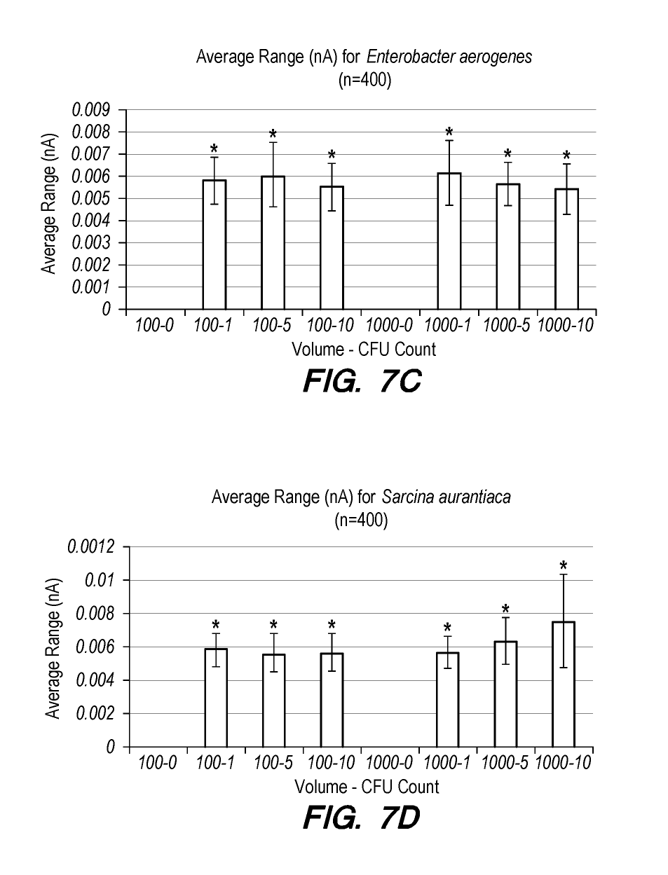

50 trials were run for each variable group, with 15 seconds per trial. The range of these trials were calculated and compared in graphs in FIGS. 7A-7D. FIGS. 7A-7D display the average amperage in nanoamperes (nA) range output by different respective biosensors at different water volumes (100 ml. and 1000 ml.) and CFU levels (1, 5 and 10 CFUs). FIG. 7A shows biosensor output detecting the presence of Escherichia coli. FIG. 7B shows biosensor output detecting the presence of Vibrio fischeri. FIG. 7C shows biosensor output detecting the presence of Enterobacter aerogenes. FIG. 7D shows biosensor output detecting the presence of Sarcina aurantiaca.

It was found that in groups where no bacteria contaminated the water, the biosensor outputted a constant value, and therefore the range was equal to zero. However, in water in which there was microorganism contamination, there existed a range of the values that was not equal to zero. This varying current and range of values signals the presence of these specific bacteria, as the released analytes bound with the receptor sites on the enzymes, which allowed for the freeing of electrons.

The average range of the current that was outputted by the eDAQ (FIG. 6) was calculated and seen here. There was no significance found between variable groups, however, there was a p value<0.001 which shows that while these biosensors were not able to directly quantify the number of CFUs present in a sample, it was able to definitively confirm the presence of bacteria.

Statistical Analysis

After running a One-Way ANOVA followed by a post-hoc scheffe test, there was no statistical significance between variable groups containing various CFU counts. However, a p value of <0.001 was discovered between the control group with solely distilled water and all other variable groups containing bacteria. This shows that while these biosensors did not appear to have the ability to quantify the number of bacterial CFU's present in a sample based on the threshold it was tested at, these biosensors were capable of definitively signaling the presence of bacteria through a changing electrical current.

Phase 2

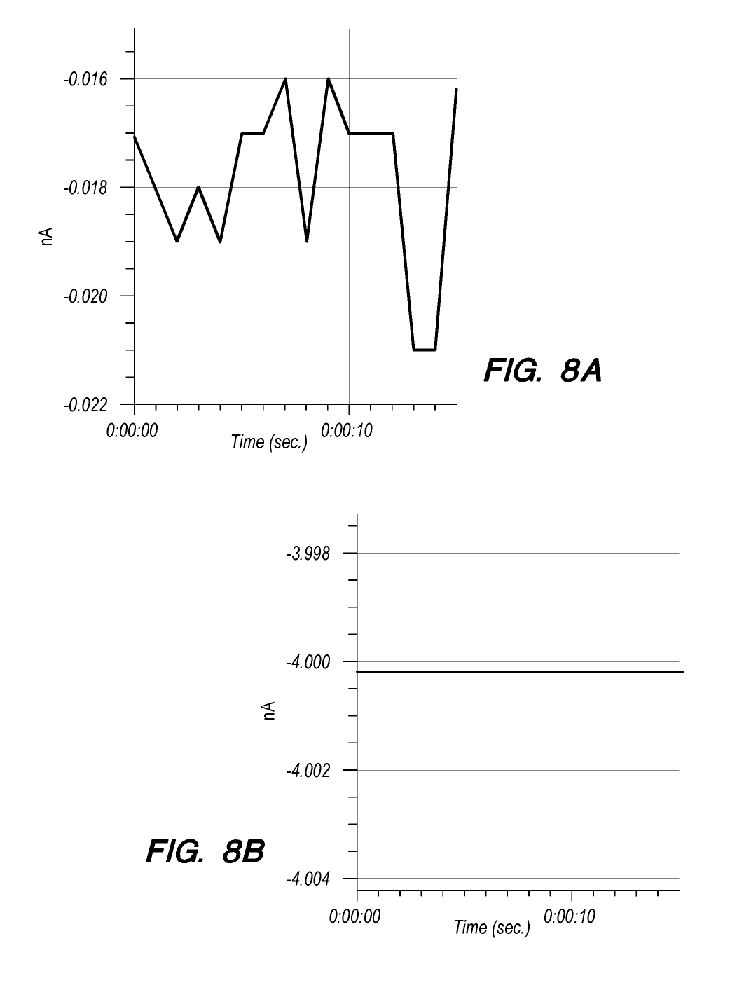

These graphs show that the purification unit had success in eliminating bacterial presence. The changing current (nA) in the graph in FIG. 8A confirms that bacteria are present in the sample within a period of 15 seconds, and after the activation of the purification unit, the current flatlines, designating that the bacteria have been removed from the sample within a period of 15 seconds. (FIG. 8B).

Discussion

Phase One:

The purpose of the first phase of this study was to create graphene biosensors (such as example biosensor devices 100) that were capable of detecting the presence of prevalent waterborne pathogens such as S. typhi, V. cholerae, S. dysenteriae, and E. coli, which are the bacteria that cause Salmonella, Cholera, and Shigellosis, and E. coli, respectively. These biosensor devices described by inventors here successfully detected the presence of these microorganisms at distinctly low levels in varying volumes of water and a rapid time frame. These biosensor devices improve greatly on current methods of bacterial detection. PCR, a traditional method of measuring bacterial contamination in water, requires a thermal cycler which can cost thousands of dollars, constant hands on work for 1-2 days, and prior training before use. PCR and traditional plating methods have detection limits from 100 to 1000 CFUs; meanwhile, the World Health Organization deemed that even 1 CFU of bacteria in 100 mL of water was unsafe (WHO). See, Deshmukh, Rehan A., Kopal Joshi, Sunil Bhand, and Utpal Roy. “Recent Developments in Detection and Enumeration of Waterborne Bacteria: A Retrospective Minireview.” MicrobiologyOpen 5.6 (2016): 901-22. Web. These biosensor devices are drastically more rapid, sensitive, inexpensive, and user-friendly. The inventors found these biosensor devices are capable of detecting at least 1 CFU of each bacteria in 1 L of water instantaneously.

When a sample is pipetted onto the graphene biosensors in contact with the ammeter, the presence of a changing electric current can be used to signal the presence of bacteria. When analytes that are released during bacterial respiration come into contact with its designated enzyme, it's catalyzed into hydrogen peroxide and pyruvate. The hydrogen peroxide is then oxidized through the applied voltage given by the ammeter, which frees electrons that the graphene harnesses in order to generate an electric current. As seen in the graphs of FIGS. 6A and 6B, the EDAQ ammeter graphed the change in nanoamps outputted by the biosensor over 15 seconds. FIG. 6A shows that there was no change in current outputted by the biosensor when uncontaminated distilled water was placed on it, as no analytes were present from bacterial respiration to be converted into electrical stimuli. However, FIG. 6B shows the results of a sample containing 1 CFU of S. aurantiaca. There is a fluctuating output, as the analytes come into contact with the enzyme and free electrons. The analytes do not all couple with the enzyme at the same time, therefore, electrons are not all freed at one time. Additionally, bacteria respire at different rates which causes the level of analytes present in a water sample to vary over a time interval. See, Jahnke, Richard A. “Quantifying the Role of Heterotrophic Bacteria in the Carbon Cycle: A Need for Respiration Rate Measurements.” Wiley Online Library, Limnology and Oceanography, Mar. 1995.

In a further advantage, the biosensor devices described here ensure specificity due to the unique nature of each of the bacterial analytes that are released during respiration. This prevents false positives from occurring. For example, the enzyme immobilized onto the Shigella biosensor was 3-Phosphate Glycerol oxidase. In order for an electrical change to occur, the specific analyte 3-Phosphate Glycerol must come into contact with the biosensor. Shigella is the only waterborne bacteria that would trigger this electrical current change, and thus, these biosensors are specific and capable of not only detecting, but identifying each bacteria present in the water.

The data received in this study proved that the biosensors were able to detect and signal the presence of bacteria through the immobilization of enzymes onto the graphene substrates. Enzymes can be immobilized onto graphene through π-π interactions, which is a type of noncovalent bond that occurs between two aromatic molecules. See, Hunter, Christopher A., and Jeremy K. M. Sanders (1990) “The Nature of .pi.-.pi. Interactions.” J. Am. Chem. Soc. Journal of the American Chemical Society 112.14: 5525-534. Web. Because graphene and 1-pyrenebutyric acid are both aromatic, meaning that they contain hexagonal benzene rings in their molecular structure, the parallel, aromatic rings align themselves with each other when 1-pyrenebutyric acid solution is incubated onto the layer of graphene (The Graphene Supermarket). This binds the PBA onto the graphene, and the PBA is then a linker molecule which latches onto the enzymes through an amide bond that occurs between the amine group of the enzymes and the pyrene group of the pyrene butyric acid. This secures and immobilizes the enzymes onto the graphene molecules. See, Karachevtsev, Victor A., and Stepan G. Stepanian (2011) “Noncovalent Interaction of Single-Walled Carbon Nanotubes with 1-Pyrenebutanoic Acid Succinimide Ester and Glucoseoxidase.” The Journal of Physical Chemistry C 115.43: 21072-1082. Web.

When the analytes that are released during bacterial respiration come into contact with the designated enzyme that is immobilized on the graphene, a series of chemical reactions occurs that signals the presence of bacteria through a changing electric current, as seen in FIG. 6B. The analyte is catalyzed into hydrogen peroxide and pyruvate, and the hydrogen peroxide is oxidized. The oxidation of hydrogen peroxide frees electrons that the graphene is able to harness due to its conductive properties to generate an electric signal. See, Karachevtsev, Victor A., and Stepan B., Stepanian (2011) “Noncovalent Interaction of Single-Walled Carbon Nanotubes with 1-Pyrenebutanoic Acid Succinimide Ester and Glucoseoxidase.” The Journal of Physical Chemistry C 115.43: 21072-1082. Web. For example, V. fischeri produces the metabolite α-D-Galactose-1 as a byproduct of respiration. When the metabolite comes into contact with galactose oxidase, the enzyme catalyzes the analyte into hydrogen peroxide and pyruvate. The hydrogen peroxide is then oxidized through the applied voltage of the ammeter, which frees electrons that graphene harnesses in order to generate a current measurable by the ammeter. See, Putzbach, William, and Niina Ronkainen (2013) “Immobilization Techniques in the Fabrication of Nanomaterial-Based Electrochemical Biosensors: A Review.” Sensors 13.4: 4811-840. Web. Because bacteria are respiring at different rates, the concentration of analytes in the sample vary, which causes the current produced by the biosensor to vary as presented by FIG. 6B. Therefore, when water was contaminated with the specific bacteria designated to the enzyme, the EDAQ ammeter measured a varying current value that signaled the presence of bacteria in water. When bacteria are not present in water, no metabolites are being produced by bacterial respiration. Therefore, no analytes are freeing electrons, which cause the current to flatline as seen in FIG. 6A.

The biosensor devices were unable to quantify the exact number of bacteria cells in the sample, however, this relationship was found in Labroo's study (2013) in a different context, in which there existed a threshold that the lactate concentration had to surpass in order to create a significantly different electrical output. See, Labroo, Pratima, and Yue Cui (2013) “Flexible Graphene Bio-nanosensor for Lactate.” Bio sensors and Bioelectronics 41: 852-56. Web. This suggests that if these biosensors were to be tested with higher levels of bacteria, and thus a higher concentration of analytes, a correlation would exist between the electrical output and the number of CFU's that exist in the sample. Importantly, however, these biosensors had the capability of identifying low levels of bacterial contaminants, which has the potential to prevent future outbreaks of bacterial infections throughout the world.

Phase Two

The purpose of the second phase of the inventors' study was to develop a mechanized purification unit (such as unit 302) that could purify water contaminated with bacteria, creating potable and usable water. Using the EDAQ ammeter and biosensors, it was confirmed that the engineered Arduino-programmed micropipette purification unit had the ability to decontaminate water that contained bacterial pathogens, thus fulfilling the purpose of this study. Current methods for purification are costly, take a long period of time, and poses risk for further contamination as water must be shipped out to external facility to be treated. This purification system allows bacteria to be eliminated from water directly within the water source in a cheap, safe manner.

When the biosensors confirm the presence of bacteria in water via a varying current, the purification unit can be activated to eliminate the bacteria. The rotation code adapted in this study can be activated on the two Arduino boards. This would allow for stepper motors to rotate, and push forward rails on rail mounts that push forward the plungers of micropipettes, releasing sodium hydroxide and hydrogen peroxide into the water. These two chemicals can lead to the generation of hydroxyl radicals, from the degradation of H2O2 into 2OH., that mark a very effective, but safe method by which water can be purified. Hydroxyl radicals uncouple bonds that exist in organic material, combining with hydrogen and carbon atoms to form water and carbon dioxide. The radicals will interact with the organic material into all the bacteria have been degraded, whereupon sodium bicarbonate can be added into the water in order stop the functioning of the radicals, and allow for complete elimination of bacteria in water. See, Ray, M. B., J. Paul Chen, and Lawrence K. Wang (2006) “Advanced Oxidation Processes.” Advanced Physicochemical Treatment Processes: 463-81. Web. The biosensors can be used in order to confirm the success of the purification unit in eliminating bacteria, as it can monitor bacterial presence regardless of any other substance that may exist in water as the enzymes immobilized on the biosensors only free electrons when the specific correlate analyte binds with the enzyme. When the bacteria are eliminated, there are no analytes being produced in the water, and therefore the current read by the ammeter remains at a constant value, which shows that the purification unit had success in eliminating bacterial presence. See, Huang, Yinxi, et al. “Nanoelectronic Biosensors Based on CVD Grown Graphene.” Nanoscale, The Royal Society of Chemistry, 11 Jun. 2010.

The purification unit in this study consisted of several features that greatly improve upon current methods of purification. For one, the purification unit had the dimensions of 44 cm×21 cm×0.5 cm. This size is much smaller than current municipal water treatment facilities, and therefore, upon implementation in a real-world situation, there exists no real size constraints, allowing for the usage of this on-site, immediate response system. Additionally, the purification system construction was much cheaper, even if scaled in size and volume, than that of a water treatment plant. The unit had a construction cost of about $150, whereas a water treatment plant can cost $500,000-$1.5 million. See, K. Marhsall, “How Much Does a Wastewater Treatment System Cost? (Pricing, Factors, Etc.),” Samco Tech, 16 May 2016. Moreover, the purification unit in this study that was created allowed for cost-effective, on-site bacteria elimination that marked a critical improvement on past methods of water purification. Compare, “Treatment Plant—Water Treatment Program—Water and Waste—City of Winnipeg.” Winnipeg. N.p., 27 Jan. 2017. Web. 22 Jun. 2017.

One area of this study that could be improved upon was the use of water-based conductive paint. Working in a high school laboratory, silver-based paint could not be utilized due to its low flash point. Therefore, water based paint was used as an alternative; however, this allows only a small volume of a sample to be pipetted onto the biosensor rather than immersing the entire substrate in a water source. If this study were to be replicated in a professional laboratory in which hydrophobic silver-based paint could be utilized, the procedure to manually transfer the sample onto the biosensor could be eliminated. The biosensors could then be simply placed in a water source, such as a well, and able to continually monitor the presence of bacteria. In spite of this limitation, however, it is important to recognize that this system had the capabilities of detecting 1 CFU of the four major bacterial contaminants of water in 1 second, and purifying water contaminated with bacterial in a rapid manner.

The biosensor devices in examples developed by the inventors and tested in this study mark important improvements on current tenets of water detection and purification. These biosensor devices were capable of detecting bacterial presence in less than 1 second, which is far more rapid than the current methods of bacterial detection and could discern lower levels of bacteria (1 CFU) than conventional methods, such as PCR and colony counting. Additionally, the mechanized purification unit was successful in eliminating bacterial presence in water in a more rapid, cost effective parameter. Finally, not only do these devices improve upon the functionality of detection and purification devices, they are also user friendly and can be implemented easily in the developing and developed world.

In conclusion, this study yielded the creation of sensors that had the ability to detect model organisms for E. coli, Salmonella, Shigella, and Cholera. Working in conjunction with these biosensors, a mechanized purification system could monitor and purify microorganism presence in order to sanitize water sources contaminated with bacterial pathogens. This system could potentially eliminate the threat of waterborne diseases and greatly expand sanitary water resources throughout the world.

### Further Embodiments and Example Implementations

The Brief Summary and Abstract sections may set forth one or more but not all exemplary embodiments of the present invention as contemplated by the inventor(s), and thus, are not intended to limit the present invention and the appended claims in any way.

Embodiments of the present invention have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed. The breadth and scope of the present invention should not be limited by any of the above-described exemplary embodiments.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

What is claimed is:

1. A rapid, highly sensitive bacteria contaminated water detection and purification system, comprising:
a biosensor having a sensing region coupled between two terminals wherein the sensing region includes an oxidase enzyme immobilized on graphene such that in the presence of bacteria respiration in contaminated water at the sensing region a current is generated between the two terminals;
a detector that detects the generated current between the two terminals and generates a signal indicative of the presence of the bacteria in the contaminated water; and
a purification unit that injects one or more fluids in the contaminated water to treat the contaminated water and obtain potable water.

2. The system of claim 1, wherein the oxidase enzyme immobilized on graphene responds to analytes generated from respiration carried out by one of the following bacteria: S. typhi, V. cholerae, E. coli, or Shigella.

3. The system of claim 1, wherein the oxidase enzyme immobilized on graphene comprises at least one of the following oxidase enzymes: glucose oxidase, glycerol 3-phosphate oxidase, galactose oxidase, or lactate oxidase.

4. The system of claim 1, wherein the oxidase enzyme immobilized on graphene further comprises pyrenebutryic acid (PBA) bonded onto the graphene.

5. The system of claim 1, wherein the detector detects the generated current between the two terminals and generates the signal indicative of the presence of the bacteria in the contaminated water in less than fifteen seconds.

6. The system of claim 1, wherein the detector detects the generated current between the two terminals and generates the signal indicative of the presence of the bacteria in the contaminated water in about one second.

7. The system of claim 1, wherein the detector detects the generated current between the two terminals and generates the signal indicative of the presence of the bacteria in the contaminated water at a detection limit of less than 100 CFUs per 100 ml. of water.

8. The system of claim 1, wherein the detector detects the generated current between the two terminals and generates the signal indicative of the presence of the bacteria in the contaminated water at a detection limit of about one CFU per liter of water.

9. The system of claim 1, wherein the detector comprises an ammeter that detects varying current indicative of the presence of bacteria respiration.

10. The system of claim 9, wherein the ammeter detects varying current in an average range of about 0.001 to 0.012 nanoamperes (nA).

11. The system of claim 1, wherein the one or more fluids injected by the purification unit comprise one or more of hydrogen peroxide or sodium hydroxide.

12. The system of claim 1, wherein the purification unit further comprises one or more microprocessor controlled ejection units to control the delivery of respective fluids into the contaminated water.

13. The system of claim 12, wherein each microprocessor controlled ejection unit comprises:
a microprocessor;
a stepper motor coupled to an ejection unit that controls the amount of respective fluid injected into the contaminated water for treatment; and
a servo-controller coupled between the microprocessor and the stepper motor, wherein the microprocessor generates signals to drive the servo-controller, and the servo-controller activates the stepper motor to control the ejection unit.

14. The system of claim 1, further comprising:
a battery; and
a housing for supporting the battery, the biosensor, the detector and the purification unit, wherein the housing has a size suitable to be hand-held, whereby a user can hold the housing and insert the biosensor or the purification unit to contact the contaminated water.

15. A biosensor device for detecting bacteria contaminated water comprising:
a graphene or graphite layer;
an oxidase enzyme layer immobilized on the graphene or graphite layer;
a base that supports the graphene or graphite layer; and
first and second conductive terminals coupled to the oxidase enzyme layer at different locations, wherein a current is generated between the first and second terminals when bacteria respiration in the contaminated water produces analytes which contact enzymes in the oxidase enzyme layer.

16. The biosensor device of claim 15, wherein the oxidase enzyme layer immobilized on the graphene or graphite layer responds to analytes generated from respiration carried out by one of the following bacteria: S. typhi, V. cholerae, E. coli, or Shigella, and generates a varying current between about 0.001 to 0.012 nanoamperes.

17. The biosensor device of claim 15, further comprising:
a detector that detects the generated current between the first and second terminals and generates a signal indicative of the presence of the bacteria in the contaminated water in less than fifteen seconds and at a detection limit of less than 100 CFUs per 100 ml. of water.

18. The biosensor device of claim 15, further comprising:
a detector that detects the generated current between the first and second terminals and generates a signal indicative of the presence of the bacteria in the contaminated water in about one second and at a detection limit of about one CFU per liter of water.

19. The biosensor device of claim 15, further comprising first and second layers of carbon epoxy coupled to the respective first and second terminals.

20. A system for detecting bacteria contaminated water comprising:
an array of biosensor devices for detecting different respective bacteria in the contaminated water including the following bacteria: S. typhi, V. cholerae, E. coli, and Shigella,
wherein each respective biosensor device in the array includes:
a respective graphene layer;
a respective oxidase enzyme layer immobilized on the respective graphene layer; and
a respective base that supports the respective graphene layer; and
a respective pair of conductive terminals coupled to the respective oxidase enzyme layer at different locations, wherein a current is generated between the pair of terminals when respiration by respective bacteria in the contaminated water produces analytes which contact enzymes in the respective oxidase enzyme layer.
